SEQUENCE LISTING (96 sequences)

SEQ ID NO: 1 - DNA, 60 bp, Mus musculus
atgggtctcaacccccagctagttgtcatcctgctcttctttctcgaatgtaccaggagc

SEQ ID NO: 2 - protein, 20 aa, Mus musculus
MGLNPQLVVILLFFLECTRS

SEQ ID NO: 3 - DNA, 60 bp, Mus musculus
atgagaaggatgcttctgcacttgagtgttctgactctcagctgtgtctgggccactgcc

SEQ ID NO: 4 - protein, 20 aa, Mus musculus
MRRMLLHLSVLTLSCVWATA

SEQ ID NO: 5 - DNA, 72 bp, Mus musculus
atgaagttcctctctgcaagagacttccatccagttgccttcttgggactgatgctggtgacaaccacggcc

SEQ ID NO: 6 - protein, 24 aa, Mus musculus
MKFLSARDFHPVAFLGLMLVTTTA

SEQ ID NO: 7 - DNA, 69 bp, Mus musculus
atgtgtcaatcacgctacctcctctttttggccacccttgccctcctaaaccacctcagtttggccagg

SEQ ID NO: 8 - protein, 23 aa, Mus musculus
MCQSRYLLFLATLALLNHLSLAR

SEQ ID NO: 9 - DNA, 63 bp, Mus musculus
atggcgctctgggtgactgcagtcctggctcttgcttgccttggtggtctcgccgccccaggg

SEQ ID NO: 10 - protein, 21 aa, Mus musculus
MALWVTAVLALACLGGLAAPG

SEQ ID NO: 11 - DNA, 69 bp, Mus musculus
atgatcttccacacaggaacaacgaagcctaccctggtgctgctttgctgtataggaacctggctggcc

SEQ ID NO: 12 - protein, 23 aa, Mus musculus
MIFHTGTTKPTLVLLCCIGTWLA

SEQ ID NO: 13 - DNA, 102 bp, Homo sapiens
atgcatccgctcctcaatcctctcctgttggcactgggcctcatggcgcttttgttgaccacggtcattgctctcacttgccttggcggctttgcctcccca

SEQ ID NO: 14 - protein, 34 aa, Homo sapiens
MHPLLNPLLLALGLMALLLTTVIALTCLGGFASP

SEQ ID NO: 15 - DNA, 69 bp, Homo sapiens
atggcctctcactcaggcccctcgacgtctgtgctctttctgttctgctgcctgggaggctggctggcc

SEQ ID NO: 16 - protein, 23 aa, Homo sapiens
MASHSGPSTSVLFLFCCLGGWLA

SEQ ID NO: 17 - DNA, 96 bp, Artificial
  Synthetic
atgggaggatccaattacactaacaattatagtaacatttctaacaactatagcaattacactaacaattatagtaacatttctaacaactatagc

SEQ ID NO: 18 - protein, 43 aa, Artificial
  Synthetic
MIFHTGTTKPTLVLLCCIGTWLATCSLSFNYTNNYSNISNNYS

SEQ ID NO: 19 - DNA, 489 bp, Mus musculus
atgatcttccacacaggaacaacgaagcctaccctggtgctgctttgctgtataggaacctggctggccacctgcagcttgtccttcggtgccccaatatcgaaggaagacttaagaactacaattgacctcttgaaacaagagtctcaggatctttataacaactatagcataaagcaggcatctgggatgtcagcagacgaatcaatacagctgccgtgtttcagcctggaccgggaagcattaaccaacatctcggtcatcatagcacatctggagaaagtcaaagtgttgagcgagaacacagtagatacttcttgggtgataagatggctaacaaacatcagctgtttcaacccactgaatttaaacatttctgtgcctggaaatactgatgaatcctatgattgtaaagtgttcgtgcttacggttttaaagcagttctcaaactgcatggcagaactgcaggctaaggacaatactacatgc

SEQ ID NO: 20 - protein, 163 aa, Mus musculus
MIFHTGTTKPTLVLLCCIGTWLATCSLSFGAPISKEDLRTTIDLLKQESQDLYNNYSIKQASGMSADESIQLPCFSLDREALTNISVIIAHLEKVKVLSENTVDTSWVIRWLTNISCFNPLNLNISVPGNTDESYDCKVFVLTVLKQFSNCMAELQAKDNTTC

SEQ ID NO: 21 - DNA, 492 bp, Mus musculus
atggcctctcactcaggcccctcgacgtctgtgctctttctgttctgctgcctgggaggctggctggcctcccacacgttgcccgtccgtttactacgaccaagtgatgatgtacagaaaatagtcgaggaattacagtccctctcgaagatgcttttgaaagatgtggaggaagagaagggcgtgctcgtgtcccagaattacacgctgccgtgtctcagccctgacgcccagccgccaaacaacatccacagcccagccatccgggcatatctcaagacaatcagacagctagacaacaaatctgttattgatgagatcatagagcacctcgacaaactcatatttcaagatgcaccagaaacaaacatttctgtgccaacagacacccatgaatgtaaacgcttcatcctgactatttctcaacagttttcagagtgcatggacctcgcactaaaatcattgacctctggagcccaacaggccaccact

SEQ ID NO: 22 - protein, 164 aa, Homo sapiens
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 23 - DNA, 801 bp, Mus musculus
atgagacctagaatgaagtattccaactccaagatttccccggcaaagttcagcagcaccgcaggcgaagccctggtcccgccttgcaaaataagaagatcccaacagaagaccaaagaattctgccatgtctactgcatgagactccgttctggcctcaccataagaaaggagactagttattttaggaaagaacccacgaaaagatattcactaaaatcgggtaccaagcatgaagagaacttctctgcctatccacgggattctaggaagagatccttgcttggcagtatccaagcatttgctgcgtctgttgacacattgagcatccaaggaacttcacttttaacacagtctcctgcctccctgagtacatacaatgaccaatctgttagttttgttttggagaatggatgttatgtgatcaatgttgacgactctggaaaagaccaagagcaagaccaggtgctactacgctactatgagtctccctgtcctgcaagtcaatcaggcgacggtgtggatgggaagaaggtgatggtgaacatgagtcccatcaaagacacagacatctggctgcatgccaacgacaaggactactccgtggagcttcaaaggggtgacgtctcgcctccggaacaggccttcttcgtccttcacaaaaagtcctcggactttgtttcatttgaatgcaagaatcttcctggcacttacataggagtaaaagataaccagctggctctagtggaggagaaagatgagagctgcaacaatattatgtttaagctctcgaaaatctaa

SEQ ID NO: 24 - protein, 266 aa, Mus musculus
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKVMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 25 - DNA, 41 bp, Artificial
  Primer
tttgaattcagcgatccgccatgatcttccacacaggaaca

SEQ ID NO: 26 - DNA, 37 bp, Artificial
  Primer
tttctcgaggccctcgaaggacaggcactgctgagga

SEQ ID NO: 27 - DNA, 574 bp, Mus musculus
tttgaattcagcgatccgccatgatcttccacacaggaacaacgaagcctaccctggtgctgctttgctgtataggaacctggctggccacctgcagcttgtccttcggtgccccaatatcgaaggaagacttaagaactacaattgacctcttgaaacaagagtctcaggatctttataacaactatagcataaagcaggcatctgggatgtcagcagacgaatcaatacagctgccgtgtttcagcctggaccgggaagcattaaccaacatctcggtcatcatagcacatctggagaaagtcaaagtgttgagcgagaacacagtagatacttcttgggtgataagatggctaacaaacatcagctgtttcaacccactgaatttaaacatttctgtgcctggaaatactgatgaatcctatgattgtaaagtgttcgtgcttacggttttaaagcagttctcaaactgcatggcagaactgcaggctaaggacaatactacatgctgagtgatgggggggggggggtgcagtgtcctcagcagtgcctgtccttcgagggcctcgagaaa

SEQ ID NO: 28 - DNA, 35 bp, Artificial
  Primer
tttctcgaggcatgtagtattgtccttagcctgca

SEQ ID NO: 29 - DNA, 35 bp, Artificial
  Primer
agaattcttgatgggtctcaacccccagctagttg

SEQ ID NO: 30 - DNA, 33 bp, Artificial
  Primer
tggatcccgagtaatccatttgcatgatgctct

SEQ ID NO: 31 - DNA, 35 bp, Artificial
  Primer
agaattcgtcatgagaaggatgcttctgcacttga

SEQ ID NO: 32 - DNA, 35 bp, Artificial
  Primer
tggatccgccttccattgcccactctgtactcatc

SEQ ID NO: 33 - DNA, 32 bp, Artificial
  Primer
agaattcatgaagttcctctctgcaagagact

SEQ ID NO: 34 - DNA, 35 bp, Artificial
  Primer
tggatccggtttgccgagtagatctcaaagtgact

SEQ ID NO: 35 - DNA, 34 bp, Artificial
  Primer
agaattcagcatgtgtcaatcacgctacctcctc

SEQ ID NO: 36 - DNA, 35 bp, Artificial
  Primer
tggatccggcggagctcagatagcccatcaccctg

SEQ ID NO: 37 - DNA, 34 bp, Artificial
  Primer
agaattcttcatggcgctctgggtgactgcagtc

SEQ ID NO: 38 - DNA, 34 bp, Artificial
  Primer
tggatccgaaggggccgtggcgaaacagttgctt

SEQ ID NO: 39 - DNA, 24 bp, Artificial
  Primer
agcttgaattcaaaggatccaaac

SEQ ID NO: 40 - DNA, 24 bp, Artificial
  Primer
tcgagtttggatcctttgaattca

SEQ ID NO: 41 - DNA, 40 bp, Artificial
  Primer
ggatcctttctcgagatggtgagcaagggcgaggagctgt

SEQ ID NO: 42 - DNA, 37 bp, Artificial
  Primer
aaagtcgacgagtccggacttgtacagctcgtccatg

SEQ ID NO: 43 - DNA, 852 bp, Artificial
  Synthetic
aagcttgaattcaaaggatcctttctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcgtcgagtctagagggcccttcgaacaaaaactcatctcagaagaggatctgaatatgcataccggtcatcatcaccatcaccattgagtttaaacc

SEQ ID NO: 44 - protein, 270 aa, Artificial
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 45 - protein, 415 aa, Artificial
  Synthetic
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 46 - protein, 408 aa, Artificial
  Synthetic
MRRMLLHLSVLTLSCVWATAMEIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEGGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 47 - protein, 486 aa, Artificial
  Synthetic
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQTGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 48 - protein, 490 aa, Artificial
  Synthetic
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 49 - protein, 406 aa, Artificial
  Synthetic
MALWVTAVLALACLGGLAAPGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPFGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 50 - protein, 438 aa, Artificial
  Synthetic
MIFHTGTTKPTLVLLCCIGTWLATCSLSFGAPISKEDLRTTIDLLKQESQDLYNNYSIKQASGMSADESIQLPCFSLDREALTNISVIIAHLEKVKVLSENTVDTSWVIRWLTNISCFNPLNLNISVPGNTDESYDCKVFVLTVLKQFSNCMAELQAKDNTTCGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 51 - DNA, 30 bp, Artificial
  Primer
gcaaatgggcggtaggcgtgtacggtggga

SEQ ID NO: 52 - DNA, 34 bp, Artificial
  Primer
tttggatccggtggccagccaggttcctatacag

SEQ ID NO: 53 - DNA, 32 bp, Artificial
  Primer
ttttggatccataaagatcctgagactcttgt

SEQ ID NO: 54 - DNA, 35 bp, Artificial
  Primer
tttggatccgctatagttgttataaagatcctgag

SEQ ID NO: 55 - DNA, 35 bp, Artificial
  Primer
tttggatccgctatagtcgtcataaagatcctgag

SEQ ID NO: 56 - DNA, 40 bp, Artificial
  Primer
caagctgcaggtggccagccaggttcctatacagcaaagc

SEQ ID NO: 57 - DNA, 37 bp, Artificial
  Primer
cttgtcccataattacactaacaattatagtaacatt

SEQ ID NO: 58 - DNA, 37 bp, Artificial
  Primer
acctggctggccacctgcagcttgtcccataattaca

SEQ ID NO: 59 - DNA, 40 bp, Artificial
  Primer
agaaaggatccgctatagttgttagaaatgttactataat

SEQ ID NO: 60 - protein, 299 aa, Artificial
  Synthetic
MIFHTGTTKPTLVLLCCIGTWLATGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 61 - protein, 328 aa, Artificial
  Synthetic
MIFHTGTTKPTLVLLCCIGTWLATCSLSFGAPISKEDLRTTIDLLKQESQDLYGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 62 - protein, 332 aa, Artificial
  Synthetic
MIFHTGTTKPTLVLLCCIGTWLATCSLSFGAPISKEDLRTTIDLLKQESQDLYNNYSGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 63 - protein, 332 aa, Artificial
  Synthetic
MIFHTGTTKPTLVLLCCIGTWLATCSLSFGAPISKEDLRTTIDLLKQESQDLYDDYSGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 64 - protein, 318 aa, Artificial
  Synthetic
MIFHTGTTKPTLVLLCCIGTWLATCSLSFNYTNNYSNISNNYSGSFLEMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLVESRGPFEQKLISEEDLNMHTGHHHHHH

SEQ ID NO: 65 - DNA, 33 bp, Artificial
  Primer
aaaggatccatggaggagccgcagtcagatcct

SEQ ID NO: 66 - DNA, 34 bp, Artificial
  Primer
tttctcgaggtctgagtcaggcccttctgtcttg

SEQ ID NO: 67 - protein, 421 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLESRGPFEQKLISEEDLNMHTGHHHHHH

SEQ ID NO: 68 - protein, 447 aa, Artificial
  Synthetic
MIFHTGTTKPTLVLLCCIGTWLATGSMEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLESRGPFEQKLISEEDLNMHTGHHHHHH

SEQ ID NO: 69 - protein, 480 aa, Artificial
  Synthetic
MIFHTGTTKPTLVLLCCIGTWLATCSLSFGAPISKEDLRTTIDLLKQESQDLYNNYSGSMEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDLESRGPFEQKLISEEDLNMHTGHHHHHH

SEQ ID NO: 70 - DNA, 34 bp, Artificial
  Primer
aaaaggatcccggcagaatcatcgagaaacctga

SEQ ID NO: 71 - DNA, 37 bp, Artificial
  Primer
tttctcgaggattttcgagagcttaaacataatattg

SEQ ID NO: 72 - DNA, 36 bp, Artificial
  Primer
aaaggatccatccaaggaacttcacttttaacacag

SEQ ID NO: 73 - protein, 294 aa, Mus musculus
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKILESRGPFEQKLISEEDLNMHTGHHHHHH

SEQ ID NO: 74 - protein, 211 aa, Artificial
  Synthetic
MIFHTGTTKPTLVLLCCIGTWLATGSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKILESRGPFEQKLISEEDLNMHTGHHHHHH

SEQ ID NO: 75 - DNA, 33 bp, Artificial
  Primer
atggacaacaccgaggacgtcatcaaggagttc

SEQ ID NO: 76 - DNA, 33 bp, Artificial
  Primer
tttgtcgacctgggagccggagtggcgggcctc

SEQ ID NO: 77 - DNA, 684 bp, Artificial
  Synthetic
atggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaagactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccaggtcgacaaa

SEQ ID NO: 78 - DNA, 39 bp, Artificial
  Primer
gctatagttgttagaaatgttactataattgttagtgta

SEQ ID NO: 79 - DNA, 39 bp, Artificial
  Primer
tttgaattcatgggaggatccaattacactaacaattat

SEQ ID NO: 80 - DNA, 63 bp, Artificial
  Primer
tttgaattcatgggaggatccaattacactaacaattatagtaacatttctaacaactatagc

SEQ ID NO: 81 - DNA, 747 bp, Artificial
  Synthetic
tttgaattcatgggaggatccaattacactaacaattatagtaacatttctaacaactatagcatggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaagactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccaggtcgacaaa

SEQ ID NO: 82 - DNA, 39 bp, Artificial
  Primer
atcctcgagcaccaccaccaccaccactgagcggccgct

SEQ ID NO: 83 - DNA, 43 bp, Artificial
  Primer
ctagagcggccgctcagtggtggtggtggtggtgctcgaggat

SEQ ID NO: 84 - protein, 251 aa, Artificial
  Synthetic
MGGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 85 - DNA, 32 bp, Artificial
  Primer
tttggatccgctcctggtacattcgagaaaga

SEQ ID NO: 86 - DNA, 32 bp, Artificial
  Primer
tttggatccggcagtggcccagacacagctga

SEQ ID NO: 87 - DNA, 32 bp, Artificial
  Primer
tttggatccggccgtggttgtcaccagcatca

SEQ ID NO: 88 - DNA, 33 bp, Artificial
  Primer
tttggatcccctggccaaactgaggtggtttag

SEQ ID NO: 89 - DNA, 33 bp, Artificial
  Primer
tttggatccccctggggcggcgagaccaccaag

SEQ ID NO: 90 - protein, 270 aa, Artificial
  Synthetic
MGLNPQLVVILLFFLECTRSGGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 91 - protein, 269 aa, Artificial
  Synthetic
MRRMLLHLSVLTLSCVWATAGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 92 - protein, 273 aa, Artificial
  Synthetic
MKFLSARDFHPVAFLGLMLVTTTAGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 93 - protein, 272 aa, Artificial
  Synthetic
MCQSRYLLFLATLALLNHLSLARGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 94 - protein, 268 aa, Artificial
  Synthetic
MALWVTAVLALACLGGLAAPGNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 95 - protein, 273 aa, Artificial
  Synthetic
MIFHTGTTKPTLVLLCCIGTWLATGSNYTNNYSNISNNYSMDNTEDVIKEFMQFKVRMEGSVNGHYFEIEGEGEGKPYEGTQTAKLQVTKGGPLPFAWDILSPQFQYGSKAYVKHPADIPDYMKLSFPEGFTWERSMNFEDGGVVEVQQDSSLQDGTFIYKVKFKGVNFPADGPVMQKKTAGWEPSTEKLYPQDGVLKGEISHALKLKDGGHYTCDFKTVYKAKKPVQLPGNHYVDSKLDITNHNEDYTVVEQYEHAEARHSGSQVEHHHHHH

SEQ ID NO: 96 - protein, 14 aa, Artificial
  Synthetic
NYTNNYSNISNNYS

(19) United States
(12) Patent Application Publication
(10) Pub. No.: US 2011/0154514 A1
(43) Pub. Date: Jun. 23, 2011

(54) Polynucleotide used for releasing recombinant protein to the outside of eukaryotic cell

(75) Inventors: Saburo Saito, Kanagawa (JP); Nobutake Akiyama, Tokyo (JP); Yuji Ohno, Tokyo (JP)

(73) Assignees: Saburo Saito, Kawagawa (JP); Nobutake Akiyama, Tokyo (JP); Oriental Yeast Co., Ltd., Tokyo (JP)

(21) Appl. No.: 12/737,072

(22) PCT Filed: Jun. 8, 2009
(86) PCT No.: PCT/JP2009/060811

§ 371 Date: Feb. 7, 2011

(30) Foreign Application Priority Data
Jun. 6, 2008 (JP) 2008-149275

(51) Int. Cl. A01K 67/00; C12P 21/06; C12N 15/63; C12N 5/10; C07K 14/00

(52) U.S. Cl. 800/8; 435/69.7; 435/69.52; 435/320.1; 435/325; 530/395

(57) ABSTRACT

This invention provides a method for efficiently producing a recombinant protein by allowing the recombinant protein to express in a eukaryotic cell and releasing the expressed recombinant protein to the outside of the cell. The invention provides a polynucleotide used for producing a recombinant protein in a host cell comprising a polynucleotide encoding a glycosylation sequence comprising a transitional endoplasmic reticulum signal sequence and the sequence represented by: Asn-X-(Thr/Ser) (wherein X is an amino acid other than proline) and a polynucleotide encoding a target protein, which would not be efficiently released to the outside of the cell even when a transitional endoplasmic reticulum signal sequence is fused. The polynucleotide releases the target protein to the outside of the host cell via sugar chain modification.

### TECHNICAL FIELD

The present invention relates to a method of ligating a signal sequence and a glycosylation sequence to a polynucleotide encoding a protein to be expressed in a eukaryotic cell and allowing the protein to express in a eukaryotic or animal cell and releasing the expressed protein to the outside of the cell. The present invention also relates to a polynucleotide, a vector, and a host cell used for such method.

### BACKGROUND ART

A protein translated by a gene plays a key role in body functions. As the gene recombination techniques have made progress, methods for target gene expression and methods for purification thereof have been developed.

It is preferable that mammalian proteins be expressed in cells in which such proteins are naturally expressed. When such cells cannot be used, however, use of cells that are phylogenetically close to the original cells is preferable from the viewpoint of protein folding or other factors. Thus, use of eukaryotic cells is preferable for the production of mammalian recombinant proteins. However, many recombinant proteins are produced and accumulated in cells. Since production of such proteins requires complicated processes of purification, protein production would be time-consuming, and mass production thereof was difficult.

Accordingly, a variety of techniques for recombinant protein production involving the use of yeast; for example, a method in which a signal sequence is ligated to a polynucleotide sequence encoding a recombinant protein to be expressed in a cell and the expressed recombinant protein is released to the outside of the cell and a variety of vectors used therefor, have been developed (see U.S. Pat. No. 5,010,003, U.S. Pat. No. 4,588,684, and WO 00/09718).

In accordance with conventional techniques, however, the efficiency for releasing, for example, a protein with a relatively high molecular weight to the outside of the cell was not sufficiently high, and development of a system that can more efficiently release a recombinant protein to the outside of the cell has been awaited.

### DISCLOSURE OF THE INVENTION

The present invention is intended to provide a method of efficiently utilizing a recombinant protein by allowing the recombinant protein to express in a eukaryotic host cell and releasing the expressed recombinant protein to the outside of the cell.

The present inventors have conducted concentrated studies regarding a method in which a recombinant protein is expressed in a host cell and released to the outside of the cell, thus allowing more efficient production of a protein than conventional techniques. The present inventors discovered that, when a recombinant protein is expressed in a host cell, sugar chain modification, such as expression of a polynucleotide encoding a transitional endoplasmic reticulum signal peptide via fusion to an upstream region of a polynucleotide encoding the target recombinant protein to be produced and ligation of a polynucleotide encoding a glycosylation sequence, would result in efficient release (i.e., secretion) of the target protein to the outside of the cell.

The present inventors discovered that use of a signal sequence exemplified as a signal sequence of a transitional endoplasmic reticulum (i.e., interleukin 4, interleukin 5, interleukin 6, interleukin 12, interleukin 13, or interleukin 31) and a glycosylation sequence of any such interleukin enables efficient release of a target protein to the outside of the host cell. This has led to the completion of the present invention.

Further, the present inventors discovered that release of the protein expressed via fusion of the transitional endoplasmic reticulum signal sequence to the artificially designed glycosylation sequence to the outside of the cell would not be influenced by the type of transitional endoplasmic reticulum signal sequence, would be significantly influenced by the presence of sugar chain modification, and would not be significantly influenced by the constitution of the peptide sequence to be released.

It was verified in the present invention that, when the target protein expressed as a fusion protein in a downstream region of the fusion protein of the transitional endoplasmic reticulum signal sequence and the glycosylation sequence is expressed in an adhesive cell (i.e., Cos-1 fibroblast) and in a suspension cell with the aid of an epidermic cell (i.e., the Freestyle 293-F cell), a sugar chain was added upon insertion of the glycosylation sequence, and the target protein was efficiently released to the outside of the cell. Such efficient protein release was observed in the fibroblast, the epidermic cell, the suspension cell, and the adhesive cell. This indicates that protein release is not influenced by cell type.

Mutant analysis demonstrated that a protein would not be efficiently released to the outside of the cell without sugar chain addition. Further, sugar-chain-degrading enzyme-based analysis demonstrated that a protein into which a glycosylation sequence had been introduced would experience modification of N-type glycosylation.

Since protein release was also accelerated by the artificially designed glycosylation sequence, the importance of the presence of the N-type sugar chain was approved, and sugar chain modification was found to be more important than the primary structure.

Two types of fluorescent proteins, murine interleukin 33 (i.e., cytokine, which would not be released in full length), and human p53 protein (i.e., nucleoprotein) were efficiently released in the culture supernatant according to the method of the present invention. This demonstrates the effects of the present invention.

As a result of comparison of transitional endoplasmic reticulum signal sequences, some differences were observed in protein release, although there were no significant differences. This indicates that the influence of the type of transitional endoplasmic reticulum signal sequence was smaller than that of glycosylation. It also indicates that what is important is the presence of the transitional endoplasmic reticulum signal sequence, with the type of such signal sequence not being significant. With the use of the signal sequence of murine interleukin 33, however, substantially no extracellular protein release occurred. Thus, the fact that the type of such signal sequence is not significant does not indicate the lack of necessity of selection of a signal sequence.

Since the human p53 protein, which is a relatively large human tumor-suppressor gene product, was released with activity, the technique of the present invention was found to be effective for the production of active proteins.

Also, a protein was released into a low-protein medium, and it facilitated protein purification. Thus, the technique of the present invention was found to be effective for protein production, including purification.

Specifically, the present invention is as follows.

[1] A polynucleotide used for producing a recombinant protein in a eukaryotic host cell comprising a polynucleotide encoding a transitional endoplasmic reticulum signal sequence and, in a downstream region thereof, a polynucleotide encoding a fusion protein of a protein with an N-type glycosylation sequence consisting of the sequence represented by: Asn-X-(Thr/Ser) (wherein X is an amino acid other than proline), the polynucleotide being used for releasing the protein to the outside of the eukaryotic host cell.

[2] A polynucleotide used for producing a recombinant protein in a eukaryotic host cell comprising a polynucleotide encoding a transitional endoplasmic reticulum signal sequence and, in a downstream region thereof, a polynucleotide encoding a fusion protein of a protein with an O-type glycosylation sequence, the polynucleotide being used for releasing the protein to the outside of the eukaryotic host cell.

[3] The polynucleotide according to [1] or [2], wherein the transitional endoplasmic reticulum signal sequence is selected from the group consisting of a signal sequence of murine interleukin 4 (SEQ ID NO: 1), a signal sequence of murine interleukin 5 (SEQ ID NO: 3), a signal sequence of murine interleukin 6 (SEQ ID NO: 5), a signal sequence of murine interleukin 12 (SEQ ID NO: 7), a signal sequence of murine interleukin 13 (SEQ ID. NO: 9), a signal sequence of murine interleukin 31 (SEQ ID NO: 11), a signal sequence of human interleukin 13 (SEQ ID NO: 13), and a signal sequence of human interleukin 31 (SEQ ID NO: 15).

[4] An expression vector comprising the polynucleotide according to any of [1] to [3], which expresses a recombinant protein and releases the expressed protein to the outside of the eukaryotic host cell.

[5] A eukaryotic host cell comprising the expression vector according to [4].

[6] An expression vector used for producing a recombinant protein in a eukaryotic host cell and for releasing the target protein to the outside of the eukaryotic host cell, which comprises a polynucleotide encoding a transitional endoplasmic reticulum signal sequence, in a downstream region thereof, a polynucleotide encoding an N-type glycosylation sequence consisting of the sequence represented by: Asn-X-(Thr/Ser) (wherein X is an amino acid other than proline), and a multicloning site capable of introducing a foreign gene encoding a target protein to be expressed into a downstream region of the polynucleotide encoding a transitional endoplasmic reticulum signal sequence and an upstream or downstream region of the polynucleotide encoding an N-type glycosylation sequence.

[7] An expression vector used for producing a recombinant protein in a eukaryotic host cell and for releasing the target protein to the outside of the host cell, which comprises a polynucleotide encoding a transitional endoplasmic reticulum signal sequence, in a downstream region thereof, a polynucleotide encoding an O-type glycosylation sequence, and a multicloning site capable of introducing a foreign gene encoding a target protein to be expressed into a downstream region of the polynucleotide encoding the transitional endoplasmic reticulum signal sequence and an upstream or downstream region of the polynucleotide encoding an O-type glycosylation sequence.

[8] A method for producing a protein comprising introducing the polynucleotide according to any of [1] to [3] into a eukaryotic host cell, culturing the eukaryotic host cell, expressing the protein encoded by the polynucleotide, releasing the expressed protein to the outside of the cell, and recovering a target protein from a cell culture supernatant.

[9] A method for producing a protein comprising introducing the expression vector according to [6] or [7] into a eukaryotic host cell, culturing the eukaryotic host cell, expressing the protein encoded by the polynucleotide, releasing the expressed protein to the outside of the cell, and recovering a target protein from a cell culture supernatant.

[10] A protein produced by the method according to [8] or [9].

[11] The protein according to [10], which is subjected to sugar chain modification via addition of an N-type glycosylation sequence consisting of the sequence represented by: Asn-X-(Thr/Ser) (wherein X is an amino acid other than proline) thereto and binding of an N-type sugar chain to the N-type glycosylation sequence.

[12] The protein according to [11], which is subjected to sugar chain modification via addition of an N-type glycosylation sequence consisting of the sequence represented by: Asn-X-(Thr/Ser) (wherein X is an amino acid other than proline) thereto, the protein not naturally undergoing sugar chain modification, and binding of an N-type sugar chain to the N-type glycosylation sequence.

[13] The protein according to [10], which is subjected to sugar chain modification via addition of an O-type glycosylation sequence thereto and binding of an O-type sugar chain to the O-type glycosylation sequence.

[14] The protein according to [13], which is subjected to sugar chain modification via addition of an O-type glycosylation sequence thereto, the protein not naturally undergoing sugar chain modification, and binding of an O-type sugar chain to the O-type glycosylation sequence.

[15] A method for producing an antibody via DNA immunization comprising introducing the polynucleotide according to any of [1] to [3] into a non-human animal, expressing a protein encoded by the polynucleotide in the animal body, and producing an antibody against the protein.

[16] A polynucleotide used for producing a recombinant protein in a host cell and releasing the target protein to the outside of the host cell via sugar chain modification, the host cell comprising a polynucleotide encoding a glycosylation sequence consisting of a transitional endoplasmic reticulum signal sequence and the sequence represented by: Asn-X-(Thr/Ser) (wherein X is an amino acid other than proline) and a polynucleotide encoding a target protein, which is not efficiently released to the outside of the host cell even upon fusion with a transitional endoplasmic reticulum signal sequence.

This description includes part or all of the contents as disclosed in the description and/or drawings of Japanese Patent Application No. 2008-149275, which is a priority document of the present application.

### BRIEF DESCRIPTION OF THE DRAWINGS

FIG. 1 is a photograph showing the results of Western blot analysis of a protein expressed in the Freestyle-293F cell with the use of an expression vector carrying polynucleotides encoding the amino acid sequences as shown in SEQ ID NOs: 45, 46, 47, 48, 49, and 50.

FIG. 2 schematically shows the locations of the signal sequence and the glycosylation sequence of mutants comprising the amino acid sequences as shown in SEQ ID NOs: 44, 60, 61, 62, 63, 50, and 64.

FIG. 3 is a photograph showing the results of Western blot analysis of a protein expressed in the Freestyle-293F cell with the use of an expression vector carrying polynucleotides encoding the amino acid sequences as shown in SEQ ID NOs: 60, 61, 62, and 63.

FIG. 4 is a photograph showing the results of Western blot analysis of a protein expressed in the Cos-1 cell with the use of an expression vector carrying polynucleotides encoding the amino acid sequences as shown in SEQ ID NOs: 44, 60, 61, 62, and 63.

FIG. 5 is a photograph showing the results of Western blot analysis of a protein expressed in the Freestyle-293F cell with the use of an expression vector carrying polynucleotides encoding the amino acid sequences as shown in SEQ ID NOs: 62 and 63.

FIG. 6 is a photograph showing the results of Western blot analysis of a protein expressed in the Freestyle-293F cell with the use of an expression vector carrying a polynucleotide encoding the amino acid sequence as shown in SEQ ID NO: 64.

FIG. 7 is a photograph showing the results of Western blot analysis of a protein expressed in the Freestyle-293F cell with the use of an expression vector carrying polynucleotides encoding the amino acid sequences as shown in SEQ ID NOs: 67, 68, and 69.

FIG. 8 is a diagram showing the sequence-selective DNA-binding ability of the p53 protein expressed in the Freestyle-293F cell with the use of an expression vector carrying polynucleotides encoding the amino acid sequences as shown in SEQ ID NOs: 67 and 69.

FIG. 9 is a photograph showing the results of purification attained by expressing a protein in the Freestyle-293F cell with the use of an expression vector carrying a polynucleotide encoding the amino acid sequence as shown in SEQ ID NO: 69 and purifying the supernatant with the use of a nickel-chelating column.

FIG. 10 is a photograph showing the results of Western blot analysis of a protein expressed in the Freestyle-293F cell with the use of an expression vector carrying polynucleotides encoding the amino acid sequences as shown in SEQ ID NOs: 73 and 74.

FIG. 11 is a photograph showing the results of Western blot analysis of a protein expressed in the Freestyle-293F cell with the use of an expression vector carrying polynucleotides encoding the amino acid sequences as shown in SEQ ID NOs: 84, 90, 91, 92, 93, 94, and 95.

### BEST MODES FOR CARRYING OUT THE INVENTION

Hereafter, the present invention is described in detail.

The signal sequence of the present invention comprises 15 to 30 amino acids that bind to the signal recognition particle (SRP) proteins (i.e., GTP-binding regulatory proteins existing in the endoplasmic reticulum), and such signal sequence is the transitional endoplasmic reticulum having a hydrophobic core mainly comprising 5 to 10 continuous hydrophobic amino acids at the N-terminus. Examples of hydrophobic amino acids include glycine, alanine, valine, leucine, isoleucine, proline, methionine, phenylalanine, tyrosine, and tryptophan. Hydrophobic amino acids in a signal sequence may be the same or different amino acids.

At the time of protein translation in the ribosome, a signal sequence is first synthesized, and the signal sequence is recognized by SRR Thereafter, translation is temporarily discontinued, SRP binds to the ribosome, and the resulting complex binds to an SRP receptor on the endoplasmic reticulum membrane. Once the signal sequence is dissociated from SRP and transferred into the endoplasmic reticulum through pores on the endoplasmic reticulum membrane, translation is restarted, and a protein enters into the endoplasmic reticulum. The signal sequence is cleaved by a peptidase in the endoplasmic reticulum, the protein is transported to the Golgi apparatus, and the protein is then released to the outside of the cell.

The signal sequence of the present invention may be composed of any sequence, provided that such sequence has activity as a signal of the transitional endoplasmic reticulum. Such signal sequence is preferably a peptide sequence, which is recognized and cleaved by a signal peptide recognition mechanism. Examples of such signal sequences include those of interleukin 4, interleukin 5, interleukin 6, interleukin 12, interleukin 13, and interleukin 31. The nucleotide sequences encoding signal sequences of murine interleukin 4, murine interleukin 5, murine interleukin 6, murine interleukin 12, murine interleukin 13, and murine interleukin 31 are shown in SEQ ID NOs: 1, 3, 5, 7, 9, and 11, respectively, and the amino acid sequences of such signal sequences are shown in SEQ ID NOs: 2, 4, 6, 8, 10, and 12, respectively. The nucleotide sequences encoding signal sequences of human interleukin 13 and human interleukin 31 are shown in SEQ ID NOs: 13 and 15, and the amino acid sequences of such signal sequences are shown in SEQ ID NOs: 14 and 16.

When a signal sequence of a protein having a signal sequence is used, a polynucleotide encoding a signal sequence may be selectively ligated to an upstream region of the target protein, and a polynucleotide encoding such protein having a signal sequence and encoding a continuous fragment containing a signal sequence may be ligated to an upstream region of the target protein.

An N-type glycosylation sequence (i.e., the N-type sugar chain modification sequence) is an N-type glycosylation sequence represented by the amino acid sequence: Asn-X-(Thr/Ser). In this formula, X represents any amino acid other than proline, and Thr/Ser represents Thr or Ser. An N-linked sugar chain binds to Asn in Asn-X-(Thr/Ser)(NXT/S) in an N-linked form. Specific examples of such sequences include NYS, NYT, NAS, NAT, NTS, and NTT. In the presence of a glycosylation sequence, a sugar chain is added to a protein in the Golgi apparatus, following migration to the endoplasmic reticulum, and the protein is released to the outside of the cell without regulation.

In addition to an N-type glycosylation sequence, an O-type glycosylation sequence can be used in the present invention. In an expression experiment upon fusion with an EGFP protein, a protein released to the outside of the cell exhibited an increase in a molecular weight via electrophoresis, which indicates O-type sugar chain modification. This demonstrates that O-type sugar chain modification also has the activity of enhancing the efficiency for extracellular release of proteins, as well as N-type sugar chain modification.

In the present invention, target proteins that are produced as recombinant proteins and released to the outside of the cells are not limited, and any proteins can be produced by the method of the present invention.

A preferable example is a protein that is not released to the outside of the cell. An example of such protein is a protein that would not experience sugar chain modification in nature. Further examples include proteins that are not released to the outside of the cell, such as a cytoplasmic protein and a nucleoprotein. A still further example is an extracellular secretion protein that is released without regulation upon conversion of a signal sequence and addition of a sugar chain sequence. A specific example of such protein is murine IL-33.

When a polynucleotide encoding a glycosylation sequence is ligated to a polynucleotide encoding a target protein to be released to the outside of the cell and the resultant is used, a polynucleotide encoding a glycosylation sequence may be ligated to an upstream region (5′-side) or a downstream region (3′-side) of a polynucleotide encoding a target protein. In such a case, the resulting sequence comprises a polynucleotide encoding a transitional endoplasmic reticulum signal sequence and, in a downstream region thereof, a polynucleotide encoding a fusion protein of a protein and an O-type glycosylation sequence added thereto.

SEQ ID NO: 17 shows the polynucleotide sequence (GSS-artificial) comprising an N-type sugar chain modification sequence ligated to a downstream region (3′-side) of a polynucleotide encoding a signal sequence of murine interleukin 31. SEQ ID NO: 18 shows the amino acid sequence (the GSS amino acid) encoded by such polynucleotide. In the amino acid sequence as shown in SEQ ID NO: 18, a glycosylation sequence is located in a downstream region of the transitional endoplasmic reticulum signal sequence. This glycosylation sequence is an artificially designed sequence, which does not exist in nature. N-type sugar chains can be added to four amino acid regions. Upon fusion of a fluorescent protein to a downstream region of such sequence, a sugar chain was added, and the protein was released to the outside of the cell. Accordingly, the glycosylation sequence is not limited to glycosylation sequences existing in nature.

Also, a polynucleotide encoding a protein having a transitional endoplasmic reticulum signal sequence and a glycosylation sequence in nature, including a polynucleotide encoding a signal sequence contained in the gene of the protein, may be used. In such a case, a continuous sequence of a polynucleotide spanning from the signal sequence of the gene of the protein to at least the glycosylation sequence of an open reading frame (ORF) encoding the protein may be ligated to an upstream region (5′-side) of a polynucleotide encoding a target protein to be produced. A polynucleotide encoding a full-length sequence containing a signal sequence of a naturally-occurring protein may be ligated to a polynucleotide encoding a target protein.

Examples of polynucleotides encoding naturally-occurring proteins comprising signal sequences and glycosylation sequences include polynucleotides encoding interleukin 13 and interleukin 31. Interleukin 13 and interleukin 31 may be derived from any animal species without limitation, and human-derived and mouse-derived interleukins can be preferably used.

Examples include a polynucleotide encoding murine interleukin 31 comprising a signal sequence as shown in SEQ ID NO: 19 and a polynucleotide encoding human interleukin 31 comprising a signal sequence as shown in SEQ ID NO: 21. Amino acid sequences encoded by such polynucleotides are shown in SEQ ID NO: 20 and SEQ ID NO: 22, respectively.

A polynucleotide that can hybridize under stringent conditions to a polynucleotide consisting of a nucleotide sequence consisting of a sequence complementary to the aforementioned nucleotide sequence, which consists of a nucleotide sequence encoding a protein having activity of the protein encoded by the polynucleotide consisting of the nucleotide sequence as shown in SEQ ID NO: 19 or 21, may also be used. The term “stringent conditions” used herein refers to, for example, 1×SSC and 0.1% SDS at 37° C., the term “more stringent conditions” refers to, for example, 0.5×SSC and 0.1% SDS at 42° C., and the term “further stringent conditions” refers to, for example, 0.2×SSC and 0.1% SDS at 65° C. As the degree of stringency for hybridization is increased, detection and isolation of DNA with higher homology can be expected. It should be noted that the aforementioned combinations of SSC, SDS, and temperature conditions are exemplary, and a person skilled in the art would be able to realize the degree of stringency equivalent to the above by adequately combining the above-mentioned and other factors that determine the degree of stringency for hybridization (e.g., polynucleotide concentration, polynucleotide length, and the duration of hybridization). In addition, a polynucleotide consisting of a nucleotide sequence having 80% or higher, preferably about 90% or higher, and more preferably about 95% or higher identity with the nucleotide sequence as shown in SEQ ID NO: 19 or 21, which is determined with the use of default parameters (default configurations) of a homology search program, such as BLAST, may be used.

When a target protein to be expressed naturally has a glycosylation sequence as exemplified by murine interleukin 33, the glycosylation sequence may be fused with a transitional endoplasmic reticulum signal sequence, so that a sugar chain is added to the glycosylation sequence, and extracellular protein release is accelerated. Under such circumstances, such glycosylation sequence existing in nature can be used. Thus, it is not always necessary to ligate a polynucleotide encoding a glycosylation sequence to an upstream or downstream region of a polynucleotide encoding a target protein. The nucleotide sequence of murine interleukin 33 is shown in SEQ ID NO: 23, and the amino acid sequence thereof is shown in SEQ ID NO: 24. In this case, such polynucleotide is within the scope of “the polynucleotide encoding a transitional endoplasmic reticulum signal sequence and, in a downstream region thereof, a polynucleotide encoding a fusion protein of a protein with an O-type glycosylation sequence or an N-type glycosylation sequence consisting of the sequence represented by: Asp-X-(Thr/Ser) (wherein X is an amino acid other than proline)” of the present invention. Also, a polynucleotide capable of hybridizing under stringent conditions to a polynucleotide consisting of a nucleotide sequence complementary to the nucleotide sequence as shown in SEQ ID NO: 23 and consisting of a nucleotide sequence encoding a protein having activity of the protein encoded by the polynucleotide consisting of nucleotide sequence as shown in SEQ ID NO: 23 may be used. In addition, a polynucleotide consisting of a nucleotide sequence having 80% or higher, preferably about 90% or higher, and more preferably about 95% or higher identity with the nucleotide sequence as shown in SEQ ID NO: 23, which is determined with the use of default parameters (default configurations) of a homology search program, such as BLAST, may be used.

The present invention includes a polynucleotide used for expressing a target protein in a host cell and releasing (secreting) the target protein to the outside of the host cell. Example of such polynucleotide include a polynucleotide encoding a transitional endoplasmic reticulum signal sequence, a polynucleotide encoding an O-type glycosylation sequence or an N-type glycosylation sequence consisting of the sequence represented by: Asp-X-(Thr/Ser) (wherein X is an amino acid other than proline), and a polynucleotide encoding the target protein. Such polynucleotide comprises, in a downstream region of a polynucleotide encoding a transitional endoplasmic reticulum signal, a polynucleotide encoding a glycosylation sequence and a polynucleotide encoding a target protein. A polynucleotide encoding a target protein and a polynucleotide encoding a glycosylation sequence may be a polynucleotide encoding a fusion protein of a target protein and a glycosylation sequence added thereto. A polynucleotide encoding such fusion protein is located in a downstream region of a polynucleotide encoding a transitional endoplasmic reticulum signal sequence. A plurality of glycosylation sequences may be ligated. Specifically, a polynucleotide used for expressing the protein of the present invention in a eukaryotic cell and releasing the expressed protein to the outside of the cell consisits of, for example, a polynucleotide encoding a transitional endoplasmic reticulum signal, a polynucleotide encoding the sequence represented by: {Asn-X-(Thr/Ser)}_{N} (wherein N is an integer of 1 to 5), and a polynucleotide encoding a target protein. Other nucleotide sequences may be included among a polynucleotide encoding a transitional endoplasmic reticulum signal, a polynucleotide encoding a glycosylation sequence, and a polynucleotide encoding a target protein.

It was actually demonstrated by the experiment involving the use of a red fluorescent protein (Dsred) and a green fluorescent protein (EGFP) that expression of proteins in an artificially designed N-type glycosylation sequence consisting of 14 amino acid residues as shown in the NYTNNYSNISNNYS sequence (SEQ ID NO: 96) in a downstream region of the transitional endoplasmic reticulum signal sequence would cause sugar chain addition and extracellular release of proteins produced along therewith would be induced.

A promoter may be operably linked to an upstream region of such polynucleotide. Any promoter may be used in the present invention, provided that such promoter is suitable for a host used for gene expression. When an yeast host is used, for example, PHO5 promoter, PGK promoter, GAP promoter, ADH promoter, or the like is preferable. When an animal host cell is used, examples of promoters include SRa promoter, SV40 promoter, LTR promoter, CMV promoter, and HSV-TK promoter. Also, an inducible promoter that is induced to function upon addition of an agent (i.e., an inducer) or other specific conditions may be used.

In the present invention, the polynucleotide used for expressing the target recombinant protein in a eukaryotic cell and secreting the expressed protein to the outside of the cell may further comprise an enhancer, a splicing signal, a poly A addition site, a selection marker, an SV40 replication origin, and the like that are known in the art.

The present invention also includes an expression cassette used for producing a recombinant protein in a host cell comprising a polynucleotide encoding a transitional endoplasmic reticulum signal sequence, in a downstream region thereof, a polynucleotide encoding a fusion protein of a protein and an O-type glycosylation sequence or an N-type glycosylation sequence consisting of the sequence represented by: Asn-X-(Thr/Ser) (wherein X is an amino acid other than proline) added thereto, a promoter, and the like.

The present invention includes a method for producing a protein comprising introducing a polynucleotide encoding a transitional endoplasmic reticulum signal sequence and, in a downstream region thereof, a polynucleotide encoding a fusion protein of a protein and an N-type glycosylation sequence consisting of the sequence represented by: Asn-X-(Thr/Ser) (wherein X is an amino acid other than proline) added thereto into a eukaryotic host cell, culturing the eukaryotic host cell, expressing a protein encoded by such polynucleotide, releasing the expressed protein to the outside of the cell, and recovering a target protein from a cell culture supernatant.

In the present invention, the polynucleotide used for expressing a target recombinant protein in a eukaryotic cell and releasing the target protein to the outside of the cell may be incorporated into a vector, and the resultant may then be introduced into a eukaryotic host cell.

Examples of eukaryotic cells used in the method of the present invention include yeast, insect, avian, amphibian, reptile, and mammalian cells.

Examples of yeast include Saccharomyces cerevisiae NA87-11A, DKD-5D, and 20B-12, Schizosaccharomyces pombe NCYC1913 and NCYC2036, and Pichia pastoris.

Examples of insect cells include Mamestra cells, such as Sf21 cells.

Examples of amphibian cells include Xenopus egg cells.

Examples of mammalian cells include: human cells, such as HEK293 cells, FreeStyle 293 cells, and FL cells; monkey cells, such as COS-7 and Vero cells; Chinese hamster cells, such as CHO and the dhfr gene-deficient CHO cells; mouse cells, such as mouse L cells, mouse AtT-20 cells, and mouse myeloma cells; and rat cells, such as rat GH3 cells.

Examples of expression vectors include pKA1, pCDM8, pSVK3, pSVL, pBK-CMV, pBK-RSV, EBV, pRS, and pYE82 vectors. If pIND/V5-His, pFLAG-CMV-2, pEGFP-N1, or pEGFP-C1 vectors are used as expression vectors, target proteins can be expressed in the form of fusion proteins to which a variety of tags, such as His, FLAG, or GFP tags, have been added.

Use of cells that can be cultured at low protein concentrations is particularly preferable since cells can be easily purified from a culture supernatant. An example of cells that can be cultured at low protein concentrations is the FreeStyle 293 cells.

A vector comprises a polynucleotide used for producing a recombinant protein in a host cell comprising a polynucleotide encoding a transitional endoplasmic reticulum signal sequence, a polynucleotide encoding an O-type glycosylation sequence or an N-type glycosylation sequence consisting of the sequence represented by: Asn-X-(Thr/Ser) (wherein X is an amino acid other than proline), and a polynucleotide encoding a target protein. The present invention includes an expression vector used for expressing such recombinant protein and releasing the expressed protein to the outside of the host cell and a host cell into which such vector has been introduced.

As a site to which a target protein is to be ligated, a multicloning site may be incorporated, and a foreign gene encoding a target protein may be incorporated into such multicloning site. In such a case, the expression vector of the present invention is used for producing a recombinant protein in a host cell, which comprises a polynucleotide encoding a transitional endoplasmic reticulum signal sequence, in a downstream region thereof, a polynucleotide encoding an O-type glycosylation sequence or an N-type glycosylation sequence consisting of the sequence represented by: Asn-X-(Thr/Ser) (wherein X is an amino acid other than proline), and a multicloning site into which a foreign gene encoding the target protein can be introduced.

A recombinant vector may be introduced into an yeast cell by any method without particular limitation, provided that DNA can be introduced into an yeast cell. Examples thereof include electroporation (Becker, D. M. et al., Methods, Enzymol., 194: 182, 1990), the spheroplast method (Hinnen, A. et al., Proc. Natl. Acad. Sci., U.S.A., 75; 1929, 1978), and a lithium acetate method (Itoh, H., J. Bacteriol., 153: 163, 1983). A recombinant vector may be introduced into an animal cell via, for example, electroporation, the calcium phosphate method, or lipofection.

According to the present invention, a eukaryotic host cell into which a vector comprising a polynucleotide used for expressing a target recombinant protein in the eukaryotic cell and secreting the target protein to the outside of the cell has been introduced may be cultured to express the target protein, and the expressed target protein can then be released to the outside of the cell (i.e., into a culture supernatant). Culture is carried out in accordance with a conventional technique used for host cell culture.

After the completion of culture, cells are separated from a supernatant in accordance with a conventional technique, and a supematant is collected. Proteins contained in the thus-obtained culture supernatant or an extract may be purified by adequately combining conventional separation and purification techniques. In comparison with proteins extracted from cells, released proteins contain less impurities or contaminants, and use of a surfactant is not necessary at the time of extraction. In this respect, such method is effective for recovery of active proteins. Examples of such techniques include treatment with the use of a modifier such as urea or a surfactant, ultrasonication, enzyme digestion, salting out or solvent precipitation, dialysis, centrifugation, ultrafiltration, gel filtration, SDS-PAGE, isoelectric focusing, ion exchange chromatography, hydrophobic chromatography, affinity chromatography, and reverse phase chromatography.

A sugar chain can be removed from the produced protein with the use of a sugar-chain-degrading enzyme. However, novel activity may be occasionally imparted to the expressed protein via glycosylation. In such a case, a glycosylation sequence is effective for preparation of a useful sugar chain protein.

The present invention includes a protein produced by the method of the present invention. Such protein is translated and it is then occasionally subjected to various types of modification in a cell. Accordingly, a modified protein is within the scope of the protein of the present invention. Examples of post-translational modification include elimination of N-terminal methionine, N-terminal acetylation, limited degradation by intracellular protease, myristoylation, isoprenylation, and phosphorylation.

The protein expression vector of the present invention comprising a polynucleotide encoding a transitional endoplasmic reticulum signal sequence and, in a downstream region thereof, a polynucleotide encoding a fusion protein of a protein and an O-type glycosylation sequence or an N-type glycosylation sequence consisting of the sequence represented by: Asn-X-(Thr/Ser) (wherein X is an amino acid other than proline) added thereto can be used for DNA immunization. Specifically, a eukaryotic cell expression vector is introduced into the muscle or skin of an animal by means of an injection or gene gun, and the expressed proteins are then released into the blood. This results in immunization, and the blood serum reacting with the target protein can be sampled. Thus, a protein expression vector of interest can be produced. Examples of animals that can be used include mice, rats, rabbits, goats, and chickens. B cells sampled from the spleen of the immunized animal may be fused with myeloma cells to prepare hybridomas, and monoclonal antibodies can then be produced.

Since the introduced proteins are released to the outside of the cell, such proteins can be used for genetic therapy and DNA vaccines. Further, cells that produce extracellular secretion proteins may be established and used for cell therapy. Also, proteins that exist in the cells and are usually recognized as autologous proteins may be released to the outside of the cell to induce immunogenicity, and the resultant may be used for preparing mouse models of autoimmune disease.

### EXAMPLES

The present invention is described in greater detail with reference to the examples below, although the technical scope of the present invention is not limited to these examples. Basic procedures regarding DNA recombinations and enzyme reactions were in accordance with the literature, “Molecular Cloning: A Laboratory Manual,” Cold Spring Harbor Laboratory, 1989. Restriction enzymes and various modification enzymes available from Invitrogen were used, unless otherwise specified. The compositions of buffers and reaction conditions for enzyme reactions were in accordance with the accompanying instructions. Method

### (1) Construction of Expression Vector

In order to prepare cDNA comprising the Kozak sequence and the EcoRI site at the N terminus and the XhoI site at the C terminus, PCR was carried out using the primer sequences (SEQ ID NO: 25 and SEQ ID NO: 26) and cDNA of the spleen cells of Balb/C mice as the template in accordance with the instructions of the Phusion PCR kit (Finnzyme, Finland). PCR was carried out at 96° C. for 2 minutes, 35 cycles of 98° C. for 15 seconds, 58° C. for 15 seconds, and 72° C. for 1 minute, and 72° C. for 1 minute. PCR was carried out under such conditions, unless otherwise specified.

The obtained PCR fragment (574 bp) was phosphorylated with the aid of T4 polynucleotide kinase (Invitrogen) in the presence of ATP (Invitrogen), the blunt-ended fragment /was cloned into the EcoRV site of the pBluescript 2 SK+ cloning vector, and a clone comprising the sequence as shown in SEQ ID NO: 27 corresponding to the relevant region of the Genbank XM_{—}132344 clone was obtained.

In order to fuse the EGFP protein and a histidine tag to the C terminus of murine IL-31, PCR was carried out using the aforementioned plasmid as a template and primer sequences (SEQ ID NO: 25 and SEQ ID NO: 28), which comprise the Kozak sequence and the EcoRI site at the N terminus and a sequence converting the termination codon at the C terminus into the XhoI site, the resultant was subcloned into the EcoRV site of the pBluescript 2 Sk+ vector, and a clone having full-length cDNA without mutation was obtained (mIL31-fus/pBSK2+).

In order to fuse the EGFP protein and a histidine tag to the C terminus of murine IL-4, 5, 6, 12, and 13, respectively, similarly, PCR was carried out using primer sequences, which comprise the Kozak sequence and the EcoRI site at the N terminus and a sequence converting the termination codon at the C terminus into the BamHI site, and cDNA of the spleen cells of Balb/C mice as the template, the resultants were subcloned into the EcoRV site of pBluescript 2 SK+ as in the case of mIL-31, and a clone having full-length cDNA without mutation was obtained (mIL-x-fus/pBSK2+ (X=4, 5, 6, 12, and 13)).

Primers as shown in SEQ ID NOs: 29 and 30 were used for murine IL-4, primers as shown in SEQ ID NOs: 31 and 32 were used for murine IL-5, primers as shown in SEQ ID NOs: 33 and 34 were used for murine IL-6, primers as shown in SEQ ID NOs: 35 and 36 were used for murine IL-12, and primers as shown in SEQ ID NOs: 37 and 38 were used for murine IL-13.

In order to conduct an analysis with the use of the enhanced green fluorescent protein (Jelly fish) (EGFP), the multiple cloning site resulting from annealing of primer sequences (SEQ ID NO: 39 and SEQ ID NO: 40) to a site between the HindIII site and the XhoI site of the mammalian expression vector (i.e., pcDNA3.1-MH-A+, Invitrogen) was modified, and the pcDNA3.1-modified+plasmid in which the positions of HindIII, EcoRI, BamHI, and XhoI had been modified was obtained.

PCR was carried out using a green fluorescent protein (EGFP) expression vector (i.e., pEGFP-C1, Clonetech) as the template and primers as shown in SEQ ID NOs: 41 and 42 to amplify cDNA of EGFP. The obtained fragment was subcloned into the EcoRV site of pBluescript 2 SK+, and clones, which did not experience mutation during PCR and oligo synthesis, were selected. The plasmid was cleaved with the BamHI and SalI restriction enzymes, and the cleaved fragment was subcloned into a site between the BamHI site and the XhoI site of the pcDNA3.1-modified+plasmid to obtain an EGFP expression vector (EGFP-H/pcDNA3.1).

This expression vector comprises the polynucleotide sequence shown below (SEQ ID NO: 43) in a downstream region of the CMV promoter and expresses the protein as shown in SEQ ID NO: 44 (EGFP-H).

As a result of such modification, the XhoI site migrates to a new location via ligation.

A fragment obtained by cleaving mIL-X-fus/pBSK2+ (X=4, 5, 6, 12, and 13) with EcoRI and BamHI was inserted into a site between EcoRI and BamHI of the prepared vector (EGFP-H/pcDNA3.1). Thus, a vector that expresses a fusion protein of full-length murine interleukin, EGFP, and a histidine tag was obtained (mIL-X-EGFPH/pcDNA3.1) (X=4, 5, 6, 12, and 13).

The mIL-4-EGFPH/pcDNA3.1 expression vector expresses the protein as shown in SEQ ID NO: 45 (mIL-4-EGFPH). The mIL-5-EGFPH/pcDNA3.1 expression vector expresses the protein as shown in SEQ ID NO: 46 (mIL-5-EGFPH). The mIL-6-EGFPH/pcDNA3.1 expression vector expresses the protein as shown in SEQ ID NO: 47 (mIL-6-EGFPH). The mIL-12-EGFPH/pcDNA3.1 expression vector expresses the protein as shown in SEQ ID NO: 48 (mIL-12-EGFPH). The mIL-13-EGFPH/pcDNA3.1 expression vector expresses the protein as shown in SEQ ID NO: 49 (mIL-13-EGFPH).

Similarly, mIL31-fus/pBSK2+ was cleaved with EcoRI and XhoI, and the resulting fragment was introduced into a site between EcoRI and XhoI of the prepared vector (EGFP-MH/pcDNA3.1-MH-A+) to obtain a vector that expresses a fusion protein of full-length murine interleukin 31, EGFP, and a histidine tag (mIL-31-EGFPH/pcDNA3.1).

The mIL-31-EGFPH/pcDNA3.1 expression vector expresses the protein as shown in SEQ ID NO: 50 (mIL-31-EGFPH).

In order to determine a region necessary for extracellular secretion of murine interleukin 31, the vector (mIL-31-EGFPH/pcDNA3.1) was subjected to modification. With the use of the mIL-31-EGFPH/pcDNA3.1 template, a primer (SEQ ID NO: 51) having a sequence within the CMV promoter region of the expression vector, and a primer shown below, a partial sequence of mIL31 was amplified via PCR, the resulting fragment was cleaved with EcoRI and BamHI, and the resultant was introduced into a site between EcoRI and BamHI of the EGFPH/pcDNA3.1 vector to prepare mutants.

The correlation between the sequences of the primers used and the resulting mutant expression vectors are as described below. SS-EGFPH/pcDNA3.1 was prepared with the use of the primers as shown in SEQ ID NO: 52 and SEQ ID NO: 51, A-EGFPH/pcDNA3.1 was prepared with the use of the primers as shown in SEQ ID NO: 53 and SEQ ID NO: 51, GSS-EGFPH/pcDNA3.1 was prepared with the use of the primers as shown in SEQ ID NO: 54 and SEQ ID NO: 51, and GSS(DD)-EGFPH/pcDNA3.1 was prepared with the use of the primers as shown in SEQ ID NO: 55 and SEQ ID NO: 51.

In order to construct signal sequences and artificial sugar chain modification sites, PCR was carried out using the mIL-31-EGFPH/pcDNA3.1 template and primers (SEQ ID NO: 51 and SEQ ID NO: 56), and fragments comprising signal sequences were obtained. Separately, PCR was carried out using the synthetic oligo DNA (SEQ ID NO: 57) template and primers (SEQ ID NO: 58 and SEQ ID NO: 59) to prepare fragments comprising artificial sugar chain modification sites. The fragments were purified, the same amounts thereof were mixed with each other, the resulting fragments were used as templates to conduct PCR with the use of the primers (SEQ ID NO: 51 and SEQ ID NO: 59), the two fragments were ligated to each other via PCR, and the resultant was amplified to prepare a cDNA fragment. After purification, the resultant was cleaved with EcoRI and BamHI, and the cleaved fragment was introduced into a site between EcoRI and BamHI of the EGFPH/pcDNA3.1 vector to prepare the SS-Art-EGFPH/pcDNA3.1 expression vector having a signal sequence and an artificial sugar chain modification site. SS-EGFPH/pcDNA3.1 expresses the protein as shown in SEQ ID NO: 60, A-EGFPH/pcDNA3.1 expresses the protein as shown in SEQ ID NO: 61, GSS-EGFPH/pcDNA3.1 expresses the protein as shown in SEQ ID NO: 62, GSS(DD)-EGFPH pcDNA3.1 expresses the protein as shown in SEQ ID NO: 63, and SS-Art-EGFPH/pcDNA3.1 expresses the protein as shown in SEQ ID NO: 64.

PCR was carried out with the use of cDNA of the human peripheral mononuclear cell as a template and primers (SEQ ID NO: 65 and SEQ ID NO: 66) to amplify cDNA encoding the human p53 protein comprising the XhoI site at the C terminus. The resulting fragment was phosphorylated, blunt-ended, and cloned into the EcoRV site of pBluescript 2 SK+. Clones free of PCR-induced mutation or mutation during primer synthesis were selected, cleaved with BamHI and XhoI, and subcloned into a site between BamHI and XhoI of pcDNA3.1-myc-His-A+ to obtain the p53 expression vector (p53H/pcDNA3.1). The p53H/pcDNA3.1 vector expresses the protein as shown in SEQ ID NO: 67 (p53 protein).

The h-p53-his/pcDNA3.1-MH-A+ vector was cleaved with BamHI and XbaI, the resulting fragment was introduced into a site between the BamHI site and the XbaI site of SS-EGFPH/pcDNA3.1 or GSS-EGFPH/pcDNA3.1, SS-p53H/pcDNA3.1 was prepared from SS-EGFPH/pcDNA3.1, and GSS-p53H/pcDNA3.1 was prepared from GSS-EGFPH/pcDNA3.1. SS-p53H/pcDNA3.1 expresses the protein as shown in SEQ ID NO: 68 (ss-p53H protein) and GSS-p53H/pcDNA3.1 expresses the protein as shown in SEQ ID NO: 69 (GSS-p53H protein). Murine interleukin 33 expression vector

PCR was carried out using mouse spleen cDNA as a template and primers (SEQ ID NOs: 70 and 71) to amplify cDNA encoding murine interleukin 33 (mIL-33) comprising the XhoI site at the C terminus. The resulting fragment was phosphorylated, blunt-ended, and cloned into the EcoRV site of pBluescript 2 SK+. Clones free of PCR-induced mutation or mutation during primer synthesis were selected, cleaved with BamHI and XhoI, and subcloned into a site between BamHI and XhoI of pcDNA3.1-myc-His-A+ to obtain the mIL-33 expression vector (mIL-33H/pcDNA3.1).

PCR was carried out using this vector as a template and primers (SEQ ID NOs: 71 and 72) to amplify a cDNA fragment encoding an mIL-33 mature region. The resulting fragment was phosphorylated, blunt-ended, and cloned into the EcoRV site of pBluescript 2 SK+. Clones free of PCR-induced mutation or mutation during primer synthesis were selected, cleaved with BamHI and XhoI, and subcloned into a site between BamHI and XhoI of SS-p53H/pcDNA3.1 to obtain an expression vector comprising mature mIL-33 in a downstream region of the mIL31 signal sequence (SS-mIL-33H/pcDNA3.1).

mIL-33H/pcDNA3.1 expresses the protein as shown in SEQ ID NO: 73 (mIL-33 protein) and SS-mIL-33H/pcDNA3.1 expresses the protein as shown in SEQ ID NO: 74 (SS-mIL-33H protein).

### Cell Culture

HEK-293 and Cos-1 cells were cultured with the use of Dulbecco's modified Eagle's medium (DMEM) (Gibco-Invitrogen), 10% fetal bovine serum (Gibco-Invitrogen) (Hyclone, Logan, Utah, U.S.A.), and penicillin/streptomycin medium (Gibco-Invitrogen) at 37° C. in the presence of 5% carbon dioxide.

The FreeStyle 293-T (FS-293T) cells (Invitrogen) were subjected to shake culture with the use of a rotary shaker in FreeStyle medium (Invitrogen) at 37° C. in the presence of 8% carbon dioxide in accordance with the instructions provided by Invitrogen.

Expression plasmids were transformed into DH5α-FT and purified using the NucleoBond Xtra Midi plasmid purification kit (Macherey-Nagel, Duren, Germany) in accordance with the instructions thereof HEK-293 and Cos-1 cells were transfected with the use of Lipofectamine 2000 (Invitrogen) and FS293-T cells were transfected with the use of 293fectin (Invitrogen) in accordance with the relevant instructions. When the blood serum is used, the low immunoglobulin fetal bovine serum (Invitrogen) was used instead in order to prevent nonspecific detection of immunoglobulin via Western blotting.

The cell culture supernatant was removed 3 days after transfection, centrifuged at 2,000 g for 5 minutes, and designated as the cell supernatant. After the supernatant was removed, HEK-293 and Cos-1 cells were washed twice with PBS in an amount the same as that of the medium, the cells were lysed with 0.5% SDS-containing PBS in an amount the same as that of the medium, and the resultant was subjected to superheating at 100° C. for 5 minutes. When FreeStyle-293T was used, cells were washed with PBS buffer via centrifugation, the cells were lysed with 0.5% SDS-containing PBS in an amount the same as that of the medium, and the resultant was subjected to superheating at 100° C. for 5 minutes.

(PBS: 137 mM NaCl, 8.1 mM Na₂HPO₄, 2.68 mM KCl, and 1.47 mM KH₂PO₄, pH 7.4) Western blotting

The cell supematant and the cell fraction extract were subjected to Western blotting with the use of the Hybond-P (PVDF) membrane (Amersham) in accordance with the instructions of the Can Get Signal kit (Toyobo Co., Ltd., Toyama, Japan). The anti-His-tag antibody (PM002, MBL, Japan) was used as the primary antibody, the anti-rabbit-IgG-HRP conjugate antibody (Cat. NA934V, GE Healthcare, U.K.) was used as the secondary antibody, and detection was carried out via exposure to Hyperfilm-ECL (Amersham) in accordance with the instructions of the ECL plus detection kit (Amersham, Oakville, Ontario, Canada).

The obtained image was converted into an electronic image with the use of an image scanner (Chem Doc XRS, Bio-Rad), and densitometry was carried out using Quantity One software included therein.

### Preparation of Art-DsredH/pcDNA3.1

PCR was carried out using a red fluorescent protein expression vector (pDsRed-Monomer-C1, Clontech) as a template and primers (SEQ ID NO: 75 and SEQ ID NO: 76). cDNA encoding a fluorescent protein was amplified, electrophoresed, and purified. Thus, cDNA encoding the nucleotide sequence as shown in SEQ ID NO: 77 comprising 684 nucleotides was obtained.

The primer as shown in SEQ ID NO: 78 was phosphorylated, and the resultant was mixed with the same amount of the primer as shown in SEQ ID NO: 79, followed by annealing and blunt-ending. Thus, cDNA as shown in SEQ ID NO: 80 was obtained. cDNA as shown in SEQ ID NO: 74 was ligated to cDNA as shown in SEQ ID NO: 80, the resultant was purified, and PCR was carried out using the purified cDNA as a template and the phosphorylated primers as shown in SEQ ID NO: 79 and SEQ ID NO: 74 to obtain cDNA as shown in SEQ ID NO: 81 comprising 747 nucleotides. Such cDNA was subcloned into the EcoRV site of pBluescript 2 SK+, and a plasmid containing an artificial glycosylation site free of mutation during primer synthesis or PCR and cDNA encoding a red fluorescent protein was obtained. The pcDNA3.1-MH-A+ vector (hwitrogen) was cleaved with EcoRV and XbaI, synthetic DNAs as shown in SEQ ID NOs: 82 and 83 were annealed thereto, a plasmid resulting from subcloning of the annealed product therein was cleaved with XhoI and EcoRI, a plasmid having the sequence as shown in SEQ ID NO: 81 was cleaved with EcoRI and SalI, and the purified cDNA comprising 747 nucleotides was subcloned to obtain a vector that expresses a red fluorescent protein containing the glycosylation sequence (i.e., Art-DsredH/pcDNA3.1).

Art-DsredH/pcDNA3.1 expresses the mIL-33 mature region as shown in SEQ ID NO: 84 (Art-DsredH protein).

Isolation of signal sequence of murine interleukin (IL-4/5/6/12/13)

In order to isolate a signal sequence of murine IL-4, PCR was carried out using the mIL-4-EGFPH/pcDNA3.1 plasmid as a template and primers (SEQ ID NO: 51 and SEQ ID NO: 85), the resultant was purified and then cleaved with HindIII and BamHI, the cleaved fragment was subcloned into a site between Hindi and BamHI of the Art-DsredH/pcDNA3.1 plasmid, and a clone having a structure of interest was selected to obtain a vector expressing a fusion protein of a signal sequence of murine IL-4, an artificial glycosylation sequence, a red fluorescent protein, and a histidine tag (SS(mIL4)-Art-DsredH/pcDNA3.1)

In order to isolate a signal sequence of murine IL-5, PCR was carried out using the mIL-5-EGFPH/pcDNA3.1 plasmid as a template and primers (SEQ ID NO: 51 and SEQ ID NO: 86), the resultant was purified and then cleaved with HindIII and BamHI, the cleaved fragment was subcloned into a site between HindIII and BamHI of the Art-DsredH/pcDNA3.1 plasmid, and a clone having a structure of interest was selected to obtain a vector expressing a fusion protein of a signal sequence of murine IL-5, an artificial glycosylation sequence, a red fluorescent protein, and a histidine tag (SS(mIL5)-Art-DsredH/pcDNA3.1).

In order to isolate a signal sequence of murine IL-6, PCR was carried out using the mIL-6-EGFPH/pcDNA3.1 plasmid as a template and primers (SEQ ID NO: 51 and SEQ ID NO: 87), the resultant was purified and then cleaved with HindIII and BamHI, the cleaved fragment was subcloned into a site between HindIII and BamHI of the Art-DsredH/pcDNA3.1 plasmid, and a clone having a structure of interest was selected to obtain a vector expressing a fusion protein of a signal sequence of murine IL-6, an artificial glycosylation sequence, a red fluorescent protein, and a histidine tag (SS(mIL6)-Art-DsredH/pcDNA3.1).

In order to isolate a signal sequence of murine IL-12, PCR was carried out using the mIL-12-EGFPH/pcDNA3.1 plasmid as a template and primers (SEQ ID NO: 51 and SEQ ID NO: 88), the resultant was purified and then cleaved with HindIII and BamHI, the cleaved fragment was subcloned into a site between HindIII and BamHI of the Art-DsredH/pcDNA3.1 plasmid, and a clone having a structure of interest was selected to obtain a vector expressing a fusion protein of a signal sequence of murine IL-12, an artificial glycosylation sequence, a red fluorescent protein, and a histidine tag (SS(mIL12)-Art-DsredH/pcDNA3.1).

In order to isolate a signal sequence of murine IL-13, PCR was carried out using the mIL-5-EGFPH/pcDNA3.1 plasmid as a template and primers (SEQ ID NO: 51 and SEQ ID NO: 89), the resultant was purified and then cleaved with HindIII and BamHI, the cleaved fragment was subcloned into a site between HindIII and BamHI of the Art-DsredH/pcDNA3.1 plasmid, and a clone having a structure of interest was selected to obtain a vector expressing a fusion protein of a signal sequence of murine IL-13, an artificial glycosylation sequence, a red fluorescent protein, and a histidine tag (SS(mIL13)-Art-DsredH/pcDNA3.1).

An expression vector containing a signal peptide of murine IL-31 (i.e., SS-EGFP-MH/pcDNA3.1-MH-A+) was cleaved with HindIII and BamHI, cDNA containing a signal peptide of murine IL-31 was purified via electrophoresis, and the resultant was subcloned into a site between HindIII and BamHI of the Art-DsredH/pcDNA3.1 plasmid to obtain a vector expressing a fusion protein of a signal sequence of murine IL-31, an artificial glycosylation sequence, a red fluorescent protein, and a histidine tag (SS(mIL31)-Art-DsredH/pcDNA3.1).

The thus-prepared expression vectors express proteins with the aid of the CMV promoter; i.e., SS(mIL4)-Art-DsredH/pcDNA3.1 expresses the protein as shown in SEQ ID NO: 90, SS(mIL5)-Art-DsredH/pcDNA3.1 expresses the protein as shown in SEQ ID NO: 91, SS(mIL6)-Art-DsredH/pcDNA3.1 expresses the protein as shown in SEQ BD NO: 92, SS(mIL12)-Art-DsredH/pcDNA3.1 expresses the protein as shown in SEQ 11) NO: 93, SS(mIL13)-Art-DsredH/pcDNA3.1 expresses the protein as shown in SEQ ID NO: 94, and SS(mIL31)-Art-DsredH/pcDNA3.1 expresses the protein as shown in SEQ ID NO: 95.

### Expression of DsRed Protein

The plasmids; Art-DsredH/pcDNA3.1 and SS (mIL-X)-Art-DsredH/pcDNA3.1 (X=4, 5, 6, 12, 13, and 31), were transfected into the Freestyle-293F and Cos-1 cells in the same manner as in the case of the EGFP protein expression vector, protein extracts of the supematant and the cell fraction were obtained, electrophoresis was carried out in 12.5% SDS-PAGE, and a histidine tag at the C terminus of the fusion protein was detected via Western blotting.

### Protein Purification

The supernatant was separated from the histidine-tagged proteins released into the FreeStyle 293 medium via centrifugation, the composition of the medium was adjusted at 50 mM Tris HCl (pH=7.4), 0.5N salt, 10 mM imidazole (Nacalai), and 0.05% Chaps (Dojindo Laboratories), and the resultant was applied to the Ni-NTA Superflow sepharose column (Cat. 30430, Qiagen) to adsorb the histidine-tagged proteins. The column was washed with a buffer containing 50 mM Tris HCl (pH=7.4), 0.5N salt, 10 mM imidazole, and 0.05% Chaps, washed with a PBS buffer, and then eluted with a buffer containing 0.5N salt and 250 mM imidazole. The eluate was concentrated with the use of an ultrafiltration concentration filter (Amicon Ultra-15, Cat. UFC901024, Millipore) and dialyzed in PBS. The obtained sample was electrophoresed in SDS-PAGE and subjected to Coomassie staining.

Activity of the p53 protein was assayed with the use of the TransAM p53 (Cat. 41196, Active Motif) in accordance with the instructions. Results

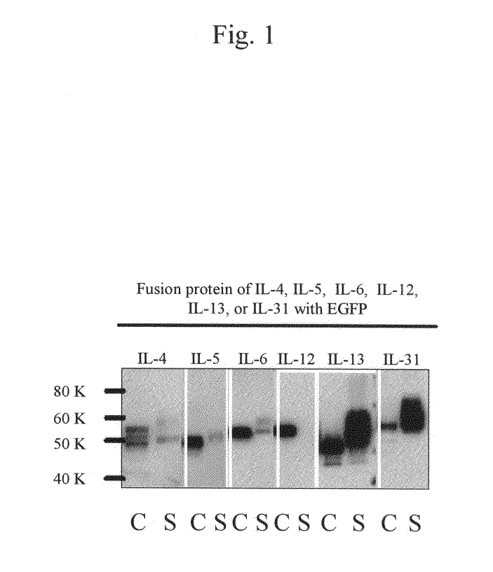

Proteins were expressed in the Freestyle-293F cells with the use of an expression vector carrying polynucleotides encoding the amino acid sequences as shown in SEQ ID NO: 45 (mIL-4-EGFPH protein), SEQ ID NO: 46 (mIL-5-EGFPH protein), SEQ ID NO: 47 L-6-EGFPH protein), SEQ ID NO: 48 (mIL-12-EGFPH protein), SEQ ID NO: 49 (mlL-13-EGFPH protein), and SEQ ID NO: 50 (mIL-31-EGFPH protein), the cell fraction samples and the supernatant fraction samples were subjected to SDS-PAGE electrophoresis, and the expressed proteins were detected via Western blotting with the use of an antibody that recognizes a histidine tag at the C terminus. The results are shown in FIG. 1. In FIG. 1, “C” represents a cell fraction and “S” represents a supernatant fraction. As shown in FIG. 1, interleukin 6 does not have an N-type glycosylation sequence, but proteins, which seem to have experienced sugar chain modification, are released into a supematant. Fusion proteins of interleukin 13 and interleukin 31 are efficiently released into a supernatant.

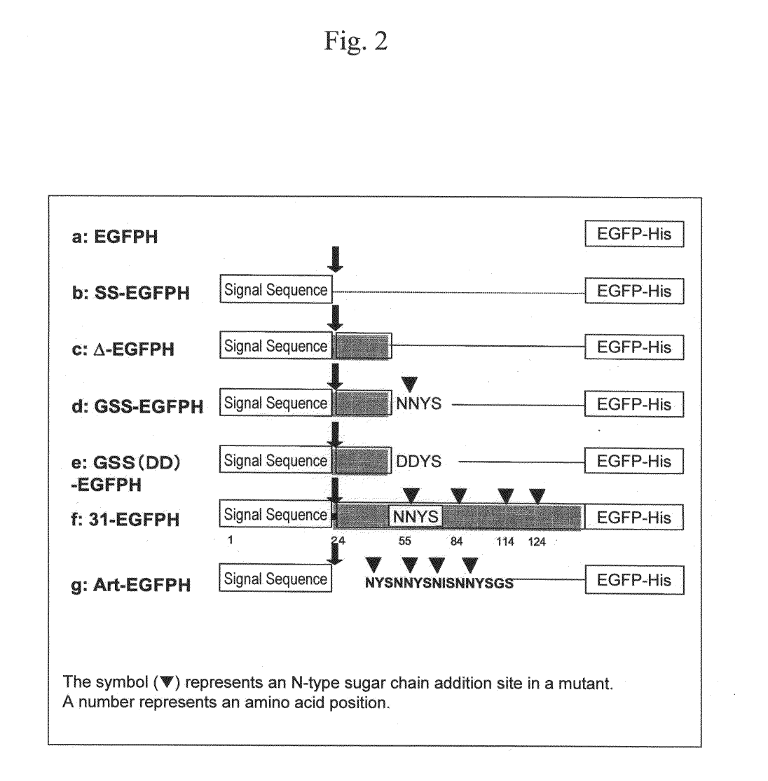

FIG. 2 schematically shows positions of signal sequences and glycosylation sequences in mutants having the amino acid sequences as shown in SEQ ID NO: 44 (EGFP-H), SEQ ID NO: 60 (SS-EGFPH/pcDNA3.1), SEQ ID NO: 61 (A-EGFPH/pcDNA3.1), SEQ ID NO: 62 (GSS-EGFPH/pcDNA3.1), SEQ ID NO: 63 (GSS (DD)-EGFPH/pcDNA3.1), SEQ ID NO: 50 (the mIL-31-EGFPH), and SEQ ID NO: 64 (SS-Art-EGFPH/pcDNA3.1). In FIG. 2, a black triangle represents an N-type sugar chain addition site in a mutant and a number represents an amino acid position.

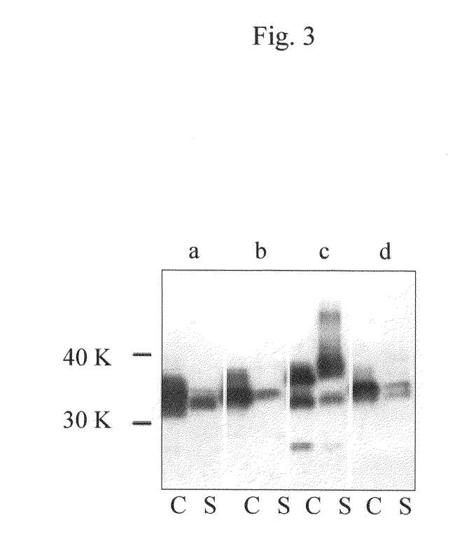

Proteins were expressed in the Freestyle-293F cells with the use of an expression vector carrying polynucleotides encoding the amino acid sequences as shown in SEQ ID NOs: 44, 60, 61, 62, and 63, the cell fraction samples and the supernatant fraction samples were subjected to SDS-PAGE electrophoresis, and the expressed proteins were detected via Western blotting with the use of an antibody that recognizes a histidine tag at the C terminus. The results are shown in FIG. 3. In the lower part of the electrophoresis photograph shown in FIG. 3, “C” represents a cell fraction and “S” represents a supernatant fraction. “a” represents SS-EGFPH (SEQ ID NO: 60), “b” represents A-EGFPH (SEQ ID NO: 61), “c” represents GSS-EGFPH (SEQ ID NO: 62), and “d” represents GSS (DD)-EGFPH (SEQ ID NO: 63). As is apparent from FIG. 3, fusion proteins are released into the medium in a particularly efficient manner in the presence of an N-type glycosylation sequence in addition to a signal sequence.

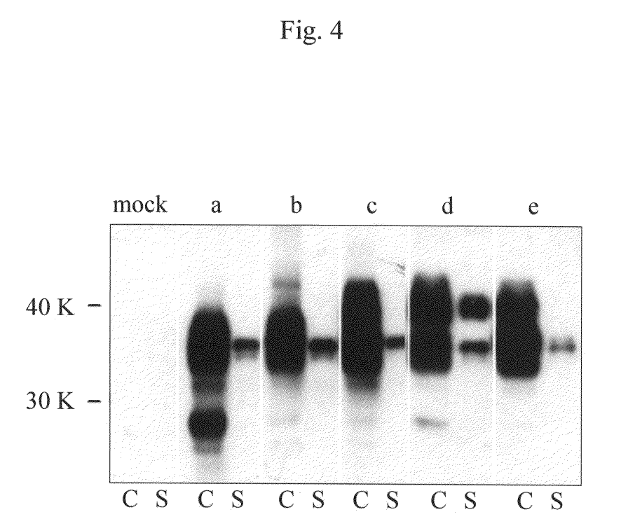

Proteins were expressed in the Cos-1 cells with the use of an expression vector carrying polynucleotides encoding the amino acid sequences as shown in SEQ ID NO: 44, 60, 61, 62, and 63, the cell fraction samples and the supernatant fraction samples were subjected to SDS-PAGE electrophoresis, and the expressed proteins were detected via Western blotting with the use of an antibody that recognizes a histidine tag at the C terminus. The results are shown in FIG. 4. In the lower part of the electrophoresis photograph shown in FIG. 4, “C” represents a cell fraction and “S” represents a supernatant fraction. “a” represents EGFPH (SEQ ID NO: 44), “b” represents SS-EGFPH (SEQ ID NO: 60), “c” represents A-EGFPH (SEQ ID NO: 61), “d” represents GSS-EGFPH (SEQ ID NO: 62), and “e” represents GSS (DD)-EGFPH (SEQ ID NO: 63). As shown in FIG. 4, the same situation as in the case shown in FIG. 3 is observed in adhesive cells, as well as in suspension cells.

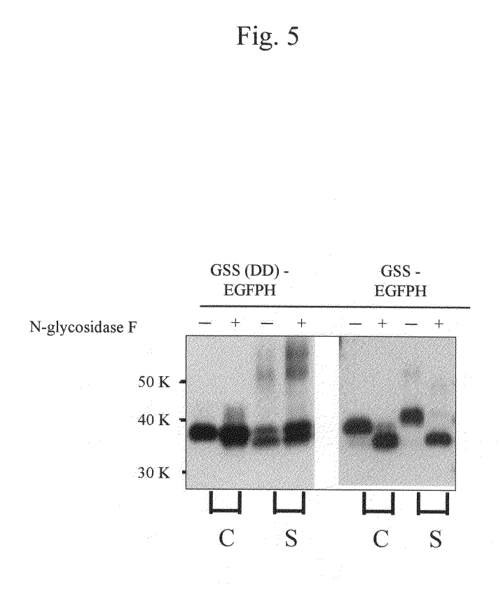

Proteins were expressed in the Freestyle-293F cells with the use of an expression vector carrying polynucleotides encoding the amino acid sequences as shown in SEQ ID NO: 62 (GSS (DD)-EGFPH) and SEQ ID NO: 63 (GSS-EGFPH), the cell fraction samples and the supernatant fraction samples were subjected to denaturation with N-glycosidase F to remove N-type sugar chains, the samples were electrophoresed in 12.5% SDS-PAGE, and a histidine tag at the C terminus was detected via Western blot analysis. The results are shown in FIG. 5. In the lower part of the electrophoresis photograph shown in FIG. 5, “C” represents a cell fraction and “S” represents a supernatant fraction. The results demonstrate that molecular weights of the GSS-EGFPH cell fraction and the GSS-EGFPH supernatant fraction are both lowered as a result of sugar chain removal. The results also demonstrate that the degree of glycosylation in the GSS-EGFPH supernatant fraction is higher than that in the GSS-EGFPH cell fraction. Since both fractions have molecular weights as predicted after sugar chain removal, it is unlikely that other protein modification has taken place.

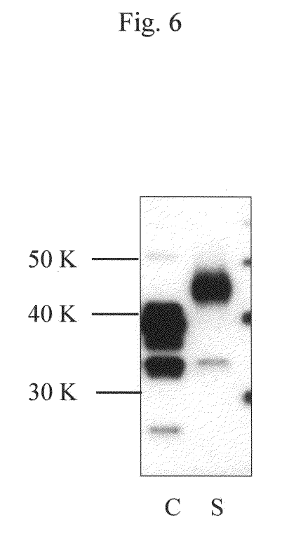

Proteins were expressed in the Freestyle-293F cells with the use of an expression vector carrying a polynucleotide encoding the amino acid sequence as shown in SEQ ID NO: 64 (GSS (ART)-EGFPH), the cell fraction sample and the supernatant fraction sample were subjected to SDS-PAGE electrophoresis, and the expressed proteins were detected via Western blotting with the use of an antibody that recognizes a histidine tag at the C terminus. The results are shown in FIG. 6. In the lower part of the electrophoresis photograph shown in FIG. 6, “C” represents a cell fraction and “S” represents a supernatant fraction. As shown in FIG. 6, sugar chain addition took place and the EGFP proteins were released even with the use of an artificially designed sugar chain modification sequence.

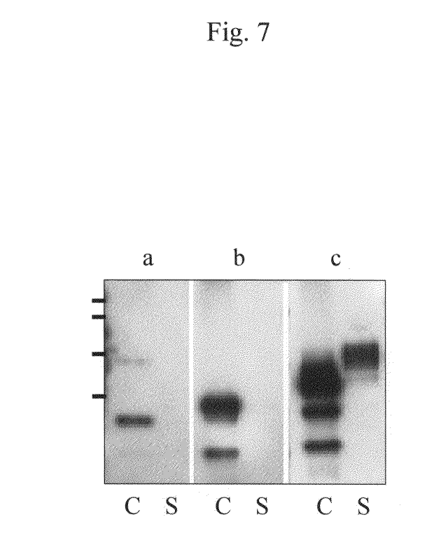

Proteins were expressed in the Freestyle-293F cells with the use of an expression vector carrying polynucleotides encoding the amino acid sequences as shown in SEQ ID NO: 67 (p53H/pcDNA3.1), SEQ ID NO: 68 (SS-p53H/pcDNA3.1), and SEQ ID NO: 69 (GSS-p53H/pcDNA3.1), the cell fraction samples and the supernatant fraction samples were subjected to SDS-PAGE electrophoresis, and the expressed proteins were detected via Western blotting with the use of an antibody that recognizes a histidine tag at the C terminus. The results are shown in FIG. 7. In the lower part of the electrophoresis photograph shown in FIG. 7, “C” represents a cell fraction and “S” represents a supernatant fraction. As shown in FIG. 7, the p53 protein was detected only in a supernatant of the GSS-p53H expression protein to which an endoplasmic reticulum signal sequence and a glycosylation sequence had been added.

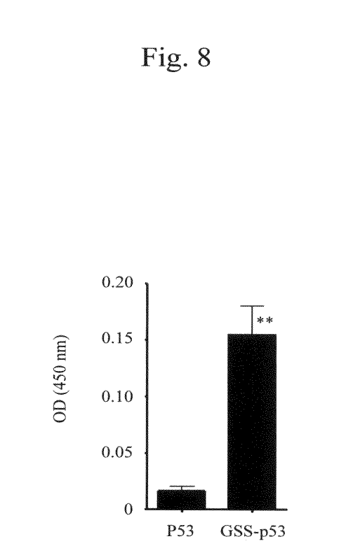

Proteins were expressed in the Freestyle-293F cells with the use of an expression vector carrying polynucleotides encoding the amino acid sequences as shown in SEQ LD NO: 67 (p53) and SEQ ID NO: 69 (GSS-p53), the sequence-selective DNA-binding capacity of the p53 proteins released in the supernatant fraction was assayed with the use of the Trans-AM kit, and the selective DNA-sequence-binding capacity was assayed at the absorption of 450 nm. The results are shown in FIG. 8. Activity of the p53 protein was assayed with the use of the TransAM p53 (Cat. 41196, Active Motif) in accordance with the instructions. As shown in FIG. 8, the active p53 proteins were released to the outside of the cell upon addition of GSS.

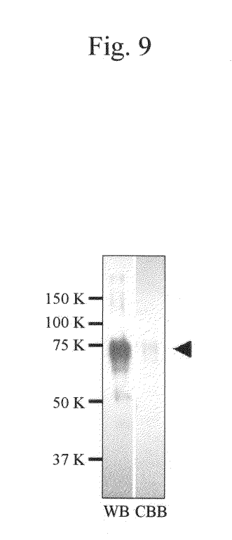

Proteins were expressed in the Freestyle-293F cells with the use of an expression vector carrying a polynucleotide encoding the amino acid sequence as shown in SEQ ID NO: 69, and the supernatant thereof was purified through a nickel chelating column. The results of analysis are shown in FIG. 9.

The supernatant was separated from the histidine-tagged proteins released into the FreeStyle 293 medium via centrifugation, the composition of the medium was adjusted at 50 mM Tris HCl (pH=7.4), 0.5N salt, 10 mM imidazole (Nacalai), and 0.05% Chaps (Dojindo Laboratories), and the resultant was applied to the Ni-NTA Superflow sepharose column (Cat 30430, Qiagen) to adsorb the histidine-tagged proteins. The column was washed with a buffer containing 50 mM Tris HCl (pH=7.4), 0.5N salt, 10 mM imidazole, and 0.05% Chaps, washed with a PBS buffer, and then eluted with a buffer containing 0.5N salt and 250 mM imidazole. The eluate was concentrated with the use of an ultrafiltration concentration filter (Amicon Ultra-15, Cat. UFC901024, Millipore) and dialyzed in PBS. The obtained sample was electrophoresed in SDS-PAGE and subjected to Coomassie staining.

“WB” indicates Western blot analysis conducted with the use of a histidine tag at the C terminus before purification and “CBB” represents an image of the purified protein subjected to Coomassie staining. An arrow head indicates the position of the p53 protein.

As shown in FIG. 9, the released proteins were exclusively purified, which was consistent with the image attained via Western blotting.

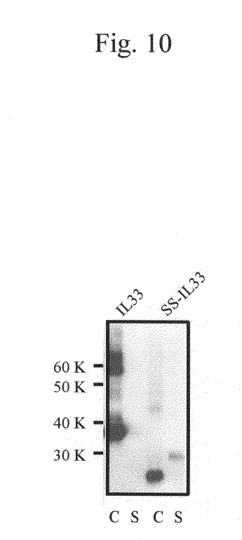

Proteins were expressed in the Freestyle-293F cells with the use of an expression vector carrying polynucleotides encoding the amino acid sequences as shown in SEQ ID NO: 73 (interleukin 33) and SEQ ID NO: 74 (a fusion protein of a signal sequence of interleukin 31 and a mature protein of interleukin 33), the cell fraction samples and the supematant fraction samples were subjected to SDS-PAGE electrophoresis, and a histidine tag added to the C terminus was detected via Western blotting. The results are shown in FIG. 10. In the lower part of the electrophoresis photograph shown in FIG. 10, “C” represents a cell fraction and “S” represents a supematant fraction.

As shown in FIG. 10, proteins were not released into the supernatant with the use of the signal sequence of interleukin 33. Upon substitution thereof with the signal sequence of interleukin 31, however, the protein of interleukin 33 was released into the supernatant with a sugar chain being added.

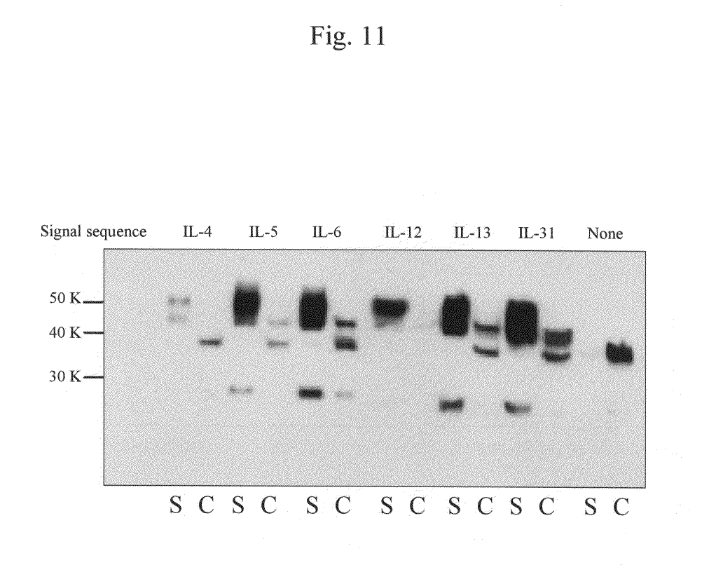

Proteins were expressed in the Freestyle-293F cells with the use of an expression vector carrying polynucleotides encoding the amino acid sequences as shown in SEQ ID NO: 84 (Art-Dsred protein), SEQ ID NO: 90 (mIL4), SEQ ID NO: 91 (mlL5), SEQ ID NO: 92 (mIL6), SEQ ID NO: 93 (mIL12), SEQ ID NO: 94 (mIL13), and SEQ ID NO: 95 (mIL31), the cell fraction samples and the supernatant fraction samples were subjected to SDS-PAGE electrophoresis, and a histidine tag added to the C terminus was detected via Western blotting. The results are shown in FIG. 11. In the lower part of the electrophoresis photograph shown in FIG. 11, “C” represents a cell fraction and “S” represents a supernatant fraction.

FIG. 11 shows a comparison of proteins to be released. Thus, the influence of the signal sequence of each murine interleukin on protein release can be observed. While a protein comprising the amino acid sequence as shown in SEQ ID NO: 84 without a signal sequence was not released, all proteins were efficiently released in the presence of a signal sequence. Also, an artificial glycosylation sequence was found to be effective for extracellular protein release.

### INDUSTRIAL APPLICABILITY

When a polynucleotide encoding a transitional endoplasmic reticulum signal sequence, a polynucleotide encoding an N-type glycosylation sequence consisting of the sequence represented by: Asn-X-(Thr/Ser) (wherein X is an amino acid other than proline) or a polynucleotide encoding an O-type glycosylation sequence, and a polynucleotide encoding a target protein are introduced into eukaryotic host cells, such cells are cultured, and proteins are expressed therein, target proteins, which have experienced sugar chain modification, are efficiently released to the outside of the host cells. By expressing the introduced proteins, which would not be originally released to the outside of the cell, and releasing the expressed proteins to the outside of the cell by such method, target proteins can be efficiently purified. In addition, the efficiency for releasing proteins, which would be originally released, to the outside of the cell can be improved by designing the sequence while taking glycosylation into consideration, and purification or other procedures thereafter can be efficiently carried out.

All publications, patents, and patent applications cited herein are incorporated herein by reference in their entirety.

1. A polynucleotide used for producing a recombinant protein in a eukaryotic host cell comprising a polynucleotide encoding a transitional endoplasmic reticulum signal sequence and, in a downstream region thereof, a polynucleotide encoding a fusion protein of a protein with an N-type glycosylation sequence consisting of a sequence represented by: Asn-X-(Thr/Ser) (wherein X is an amino acid other than proline), the polynucleotide being used for releasing the protein to the outside of the eukaryotic host cell.

2. A polynucleotide used for producing a recombinant protein in a eukaryotic host cell comprising a polynucleotide encoding a transitional endoplasmic reticulum signal sequence and, in a downstream region thereof, a polynucleotide encoding a fusion protein of a protein with an O-type glycosylation sequence, the polynucleotide being used for releasing the protein to the outside of the eukaryotic host cell.

3. The polynucleotide according to claim 1, wherein the transitional endoplasmic reticulum signal sequence is selected from the group consisting of a signal sequence of murine interleukin 4 (SEQ ID NO: 1), a signal sequence of murine interleukin 5 (SEQ ID NO: 3), a signal sequence of murine interleukin 6 (SEQ ID NO: 5), a signal sequence of murine interleukin 12 (SEQ ID NO: 7), a signal sequence of murine interleukin 13 (SEQ ID NO: 9), a signal sequence of murine interleukin 31 (SEQ ID NO: 11), a signal sequence of human interleukin 13(SEQ ID NO: 13), and a signal sequence of human interleukin 31 (SEQ ID NO: 15).

4. An expression vector comprising the polynucleotide according to claim 1, which expresses a recombinant protein and releases the expressed protein to the outside of the eukaryotic host cell.

5. A eukaryotic host cell comprising the expression vector according to claim 4.

6. An expression vector used for producing a recombinant protein in a eukaryotic host cell and for releasing the target protein to the outside of the eukaryotic host cell, which comprises a polynucleotide encoding a transitional endoplasmic reticulum signal sequence, in a downstream region thereof, a polynucleotide encoding an N-type glycosylation sequence consisting of the sequence represented by: Asn-X-(Thr/Ser) (wherein X is an amino acid other than proline), and a multicloning site capable of introducing a foreign gene encoding a target protein to be expressed into a downstream region of the polynucleotide encoding a transitional endoplasmic reticulum signal sequence and an upstream or downstream region of the polynucleotide encoding an N-type glycosylation sequence.

7. An expression vector used for producing a recombinant protein in a eukaryotic host cell and for releasing the target protein to the outside of the host cell, which comprises a polynucleotide encoding a transitional endoplasmic reticulum signal sequence, in a downstream region thereof, a polynucleotide encoding an O-type glycosylation sequence, and a multicloning site capable of introducing a foreign gene encoding a target protein to be expressed into a downstream region of the polynucleotide encoding a transitional endoplasmic reticulum signal sequence and an upstream or downstream region of the polynucleotide encoding an O-type glycosylation sequence.

8. A method for producing a protein comprising introducing the polynucleotide according to claim 1 into a eukaryotic host cell, culturing the eukaryotic host cell, expressing the protein encoded by the polynucleotide, releasing the expressed protein to the outside of the cell, and recovering a target protein from a cell culture supernatant.

9. A method for producing a protein comprising introducing the expression vector according to claim 6 into a eukaryotic host cell, culturing the eukaryotic host cell, expressing the protein encoded by the polynucleotide, releasing the expressed protein to the outside of the cell, and recovering a target protein from a cell culture supernatant.

10. A protein produced by the method according to claim 8.

11. The protein according to claim 10, which is subjected to sugar chain modification via addition of an N-type glycosylation sequence consisting of the sequence represented by: Asn-X-(Thr/Ser) (wherein X is an amino acid other than proline) thereto and binding of an N-type sugar chain to the N-type glycosylation sequence.

12. The protein according to claim 11, which is subjected to sugar chain modification via addition of an N-type glycosylation sequence consisting of the sequence represented by: Asn-X-(Thr/Ser) (wherein X is an amino acid other than proline) thereto, the protein not naturally undergoing sugar chain modification, and binding of an N-type sugar chain to the N-type glycosylation sequence.

13. The protein according to claim 10, which is subjected to sugar chain modification via addition of an O-type glycosylation sequence thereto and binding of an O-type sugar chain to the O-type glycosylation sequence.

14. The protein according to claim 13, which is subjected to sugar chain modification via addition of an O-type glycosylation sequence thereto, the protein not naturally undergoing sugar chain modification, and binding of an O-type sugar chain to the O-type glycosylation sequence.

15. A method for producing an antibody via DNA immunization comprising introducing the polynucleotide according to claim 1 into a non-human animal, expressing a protein encoded by the polynucleotide in the animal body, and producing an antibody against the protein.

16. The polynucleotide according to claim 2, wherein the transitional endoplasmic reticulum signal sequence is selected from the group consisting of a signal sequence of murine interleukin 4 (SEQ ID NO: 1), a signal sequence of murine interleukin 5 (SEQ ID NO: 3), a signal sequence of murine interleukin 6 (SEQ ID NO: 5), a signal sequence of murine interleukin 12 (SEQ ID NO: 7), a signal sequence of murine interleukin 13 (SEQ ID NO: 9), a signal sequence of murine interleukin 31 (SEQ ID NO: 11), a signal sequence of human interleukin 13(SEQ ID NO: 13), and a signal sequence of human interleukin 31 (SEQ ID NO: 15).

17. An expression vector comprising the polynucleotide according to claim 2, which expresses a recombinant protein and releases the expressed protein to the outside of the eukaryotic host cell.

18. A method for producing a protein comprising introducing the polynucleotide according to claim 2 into a eukaryotic host cell, culturing the eukaryotic host cell, expressing the protein encoded by the polynucleotide, releasing the expressed protein to the outside of the cell, and recovering a target protein from a cell culture supernatant.

19. A method for producing a protein comprising introducing the expression vector according to claim 7 into a eukaryotic host cell, culturing the eukaryotic host cell, expressing the protein encoded by the polynucleotide, releasing the expressed protein to the outside of the cell, and recovering a target protein from a cell culture supernatant.

20. A protein produced by the method according to claim 9.

21. A method for producing an antibody via DNA immunization comprising introducing the polynucleotide according to claim 2 into a non-human animal, expressing a protein encoded by the polynucleotide in the animal body, and producing an antibody against the protein.
